(19) United States
(12) Patent Application Publication
(10) Pub. No.: US 2006/0009475 A1
(43) Pub. Date: Jan. 12, 2006

(54) Quinazolinone compounds in combined modalities for improved cancer treatment

(76) Inventors: Mark Pines, Havazelet (IL); Israel Vlodavsky, Mevaseret Zion (IL); Shai Yarkoni, Kfar Saba (IL); Arnon Nagler, Jerusalem (IL)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/500,690

(22) PCT Filed: Dec. 30, 2002
(86) PCT No.: PCT/IL02/01055

§ 371 Date: Aug. 15, 2005

(30) Foreign Application Priority Data
Dec. 31, 2001 (IL) 147416

(51) Int. Cl. A61K 31/517

(52) U.S. Cl. 514/266.22

(57) ABSTRACT

The present invention provides compositions and methods for improving the effectiveness of anti-tumor treatments. The compositions of the present invention comprise quinazolinones, specifically halofuginone. In currently preferred embodiments the compositions and methods of the present invention improve the effectiveness of radiation therapy and chemotherapy, and concomitantly alleviate or prevent the damage induced by radiation therapy.

### FIELD OF THE INVENTION

The present invention relates to the field of cancer treatment, specifically to the synergistic effects obtained by the administration of quinazolinone derivatives, particularly halofuginone, in conjunction with additional anti tumor therapies.

### BACKGROUND OF THE INVENTION

Fibrosis

Clinical conditions and disorders associated with primary or secondary fibrosis are characterized by excessive production of connective tissue, resulting in destruction of normal tissue architecture and function. Fibrosis results from diverse modes of trauma including burns, surgery, infection, alcohol consumption and exposure to toxins.

Acute fibrosis is also a common adverse effect associated with cancer therapy, including radiation and chemotherapy treatments.

Radiation and fibrosis

Radiation fibrosis is an extremely severe adverse effect of ionizing radiation employed in therapy of various cancerous conditions. Fibrosis may develop as a sequel of the necessary radiotherapy and the accidental overexposures associated with the therapy. As of today, preventive or curative treatment for radiation fibrosis is not available.

Fibrosis disorders following radiation have been described in almost any tissue, including skin, lung, heart, liver and kidneys, and have shown to cause acute complications (such as bowel obstruction, severe lung injury, etc.).

Medical treatments used to overcome such acute complications resulting from radiation fibrosis were not shown to have beneficial effects. The most common method used is surgery, which is rarely successful, generally requires repeated operations, and is accompanied with poor recovery.

The clinical conditions and disorders related to radiation fibrosis are characterized by excessive production of connective tissue, resulting in the destruction of normal tissue architecture and function.

Although radiation fibrosis has been reported for many years in histopathological studies, the mechanisms of its initiation and chronic extension still remain to be resolved. Fibrosis is in fact a dynamic process, characterized by constant remodeling and long term fibroblast activation. In normal wound healing, fibroblasts are transiently activated into myofibroblasts to proliferate and deposit the collagen matrix. Feedback mechanisms then occur to down regulate cellular activities, and it has been proposed that myofibroblasts become terminally differentiated and finally disappear due to apoptosis. On the contrary, in fibrosis, the feedback regulations are not observed, and chronic, long term myofibroblast activation is sustained. One possible origin of the chronic cellular activation could be an abnormal production of stimulating factors such as cytokines and growth factors.

Recently, a new concept was proposed regarding the initiation of radiation damage, suggesting that a cascade of cytokines initiated immediately after irradiation persists for long periods of time and leads to the development of late damage.

Chemotherarpy and Fibrosis

Several cytotoxic agents commonly used in chemotherapy are known to induce fibrosis in different organs. One of the most widely reported agents is Bleomycin, which is known to induce lung fibrosis. Other agents associated with high number of fibrosis incidence include busulfan, carmustine (BCNU), and mitomycin-C.

Bleomycin is reported to induce pulmonary fibrosis in approximately 10% to 30% of treated patients, with death of 1% to 2% of patients associated with pulmonary fibrosis (Wesselius L., J. Comp. Ther. 1999:25 (5):272-277).

Intra-abdominal and retroperitoneal fibrosis have been described as secondary to intraperitoneal (IP) administration of several chemotherapeutic agents, including carboplatin, mitoxantrone and the combination of 5-fluorouracil and cisplatin (Fata et. al., Cancer 2000, June 1:88(11):2447-51).

Adriamycin, administered either in conventional or liposomal formulations, is known to induce fibrotic encapsulation of tumors that decreases the concentrations of the drug in the tumor, leading to reduced efficacy of the chemotherapy.

Halofuginone

U.S. Pat. No. 3,320,124 disclosed and claimed a method for treating coccidiosis with quinazolinone derivatives. Halofuginone, otherwise known as 7-bromo-6-chloro-3-[3-(3-hydroxy-2-piperidinyl)-2-oxopropyl]-4(3H)-quinazolinone (one of the quinazolinone derivatives), was first described and claimed in said patent to American Cyanamid, and was the preferred compound taught by said patent and the one commercialized from among the derivatives described and claimed therein. Subsequent U.S. Pat. Nos. 4,824,847; 4,855,299; 4,861,758 and 5,215,993 all relate to the coccidiocidal properties of Halofuginone.

More recently, some of the inventors of the present invention (U.S. Pat. No. 5,449,678 to Pines et. al) disclosed that these quinazolinone derivatives are unexpectedly useful for the treatment of a fibrotic condition. That disclosure provides compositions of a specific inhibitor comprising a therapeutically effective amount of a compound having the general formula I: 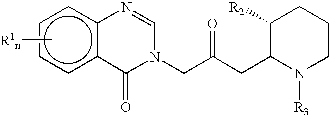
wherein: n=1-2
R₁ is at each occurrence independently selected from the group consisting of a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl; or pharmaceutically acceptable salts thereof.

The '678 patent discloses that these compounds are effective in the treatment of fibrotic conditions such as scleroderma and graft versus host disease (GVHD). Of this group of compounds, halofuginone has been found to be particularly effective.

Some of the inventors of the present invention have further disclosed in U.S. Pat. No. 5,891,879 to Nagler et al. that these compounds are effective in treating restenosis. Both conditions, namely fibrosis and restenosis are associated with excessive collagen deposition, which can be inhibited by halofuginone. Restenosis is characterized by smooth muscle cell proliferation and extracellular matrix accumulation within the lumen of affected blood vessels in response to a vascular injury (Choi et al., Arch. Surg., 1995, 130:257-261). One characteristic of such smooth muscle cell proliferation is a phenotypic alteration, from the normal contractile phenotype to a synthetic one. Type I collagen has been shown to support such a phenotypic alteration, which can be blocked by halofuginone (Choi et al., Arch. Surg., 130:257-261, 1995; U.S. Pat. No. 5,449,678).

Notably, halofuginone inhibits collagen synthesis by fibroblasts in vitro, however, it promotes wound healing in vivo (WO 01/17531 to Nagler et. al). Thus, the exact behavior of halofuginone in vivo cannot always be accurately predicted from in vitro studies.

In addition, pharmaceutical compositions comprising quinazolinone, including halofuginone, have been disclosed and claimed as effective for treating malignancies (U.S. Pat. No. 6,028,075 to Pines et. al) as well as for prevention of neovascularization (U.S. Pat. No. 6,090,814 to Nagler et. al).

The ability of halofuginone, or other related quinazolinone derivatives, to enhance the efficacy of known anti-tumor treatments, particularly radiation or chemotherapy, was neither taught or suggested in the background art. Such enhancement may reduce the dose required for successful anti-tumor treatment, leading to a reduction in the undesired adverse effects, including fibrosis.

### SUMMARY OF THE INVENTION

It is now disclosed that pharmaceutical compositions comprising quinazolinone derivatives, specifically halofuginone, can unexpectedly improve the effectiveness of anti tumor treatments, such as radiation and chemotherapy. The present invention further proposes that the synergistic effect of quinazolinone is mediated by increasing the sensitivity of tumor cells to the ionizing radiation or to the chemotherapy treatment.

According to one aspect the present invention provides a method for increasing the effectiveness of anti-tumor treatments, the method comprising the step of co-administering to a subject in need thereof a pharmaceutical composition comprising as an active ingredient a quinazolinone derivative compound having the general formula I: 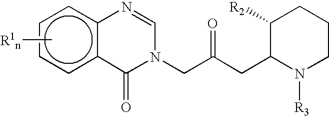
wherein: n=1-2
R₁ at each occurrence is independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof,
and at least one additional anti tumor treatment.

According to one currently preferred embodiment, the quinazolinone derivative is halofuginone.

According to one embodiment, the at least one additional anti tumor treatment administered in combination with the quinazolinone compositions of the present invention is selected from the group consisting of radiation therapy, chemotherapy, immunotherapy, hormonal therapy and genetic therapy.

According to one currently preferred embodiment the anti tumor treatment is selected from the group consisting of radiation or chemotherapy.

According to one embodiment, the chemotherapeutic agent is selected from the group consisting of topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, megestrol acetate.

According to one embodiment the co-treatment of the present invention is performed by separate administrations of each of the treatments, namely the administration of quinazolinone compositions and the administration of at least one additional anti tumor treatment.

According to another embodiment, the administration of the quinazolinone composition is essentially at the same time as the administration of the additional anti tumor treatment.

According to another embodiment when the additional anti tumor treatment is chemotherapy, co-administration of the two agents, whether as a single combined composition or in separate compositions, is also shown to act synergistically.

According to another embodiment the present invention provides a method for increasing the effectiveness of additional anti-tumor treatments, by pre-administering quinazolinone derivative compounds having the general formula I: 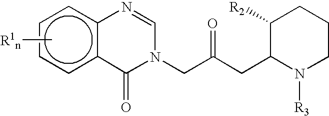
wherein: n=1-2
R₁ at each occurrence is independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof,
followed by the administration of at least one additional anti-tumor treatment.

Treatment with quinazolinones according to the present invention can be particularly effective and beneficial when administered prior to the administration of an additional anti-tumor chemotherapeutic agent or to treatment with radiation therapy. This advantage is attained by the use of halofuginone to synchronize the cells, thereby rendering them more susceptible to the subsequent anti-tumor treatment.

According to another aspect the present invention provides a combined composition for increasing the effectiveness of anti tumor treatments, comprising a quinazolinone derivative compound having the general formula 1: 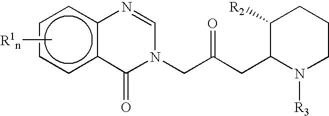
wherein: n=1-2
R₁ is a at each occurrence independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof,
further comprising at least one additional anti tumor agent.

According to one currently preferred embodiment the combined composition comprises halofuginone.

According to another embodiment, the at least one additional anti tumor agent present in combination with the quinazolinone in the compositions of the present invention is selected from the group consisting of topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, megestrol acetate.

According to yet another aspect the present invention provides the use of a quinazolinone derivative having the general formula I: 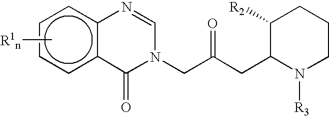
wherein: n=1-2
R₁ at each occurrence is independently a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl; or pharmaceutically acceptable salts thereof,
in preparation of a medicament for treating a tumor in combination therapy with at least one additional anti tumor treatment, thereby improving the effectiveness of the anti tumor treatment.

According to one currently preferred embodiment the quinazolinone derivative used in combined therapy is halofuginone.

According to one embodiment, the combined therapy comprises an additional known anti tumor treatment selected from the group consisting of radiation therapy, chemotherapy, immunotherapy, hormonal therapy and genetic therapy.

According to one currently preferred embodiment the combined therapy comprises an additional anti tumor treatment selected from the group consisting of radiation or chemotherapy.

According to another embodiment, the chemotherapeutic agent is selected from the group consisting of topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, megestrol acetate.

According to yet another aspect the present invention provides a method for alleviating or preventing the damage induced by radiation therapy comprising the step of administering to a subject undergoing radiation therapy a therapeutically effective amount of a quinazolinone derivative compound having the general formula I: 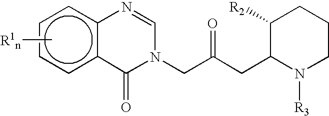
wherein: n=1-2
R₁ at each occurrence is independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof.

According to one currently preferred embodiment the quinazolinone derivative used in the method of preventing radiation damage is halofuginone.

According to another currently preferred embodiment the administration of the quinazolinine compositions of the present invention is prior to the administration of the radiation therapy.

The present invention is explained in greater detail in the description, figures and claims below.

### BRIEF DESCRIPTION OF THE FIGURES

FIG. 1 shows the effect of halofuginone on cell cycle of rabbit aortic smooth muscle cells (SMC).

FIG. 2 shows the effect of halofuginone on cell cycle of U266 cells.

FIG. 3 shows the effect of combination treatment—halofuginone (HF)+Melphalan—on the viability of U266 cells.

FIG. 4 illustrates leg contraction as a model for radiation-induced fibrosis.

FIG. 5 shows the influence of halofuginone (μg/mouse) on the contraction of irradiated mice legs.

FIG. 6 shows radiation survival curves for two human pancreatic cancer cell lines pre-treated with Halofuginone.

### DETAILED DESCRIPTION OF THE INVENTION

The combination of treatments with different modes of action in cancer therapy is currently gaining a lot of enthusiasm. Combining different modalities or even specific agents with different mechanism of action and different adverse effects, allows for better efficacy with fewer side effects.

In this context quinazolinone derivatives, preferably halofuginone, are now disclosed to improve the effect of other anti-tumor agents or treatments, either through enhancing the effect of the anti-tumor treatment or through the reduction of adverse effects associate with the treatment.

Unexpectedly, it has been found, as exemplified in detail herein below, that pharmaceutical compositions comprising quinazolinone derivatives, preferably halofuginone, can synergistically enhance the effectiveness of known anti-tumor treatments including, but not limited to, radiation therapy and chemotherapy.

Hereinafter, the term “anti tumor treatments” refers to any anti tumor treatment approved for use in a subject. The term “radiation therapy” refers to treatment of cancer through ionizing radiation, as is well known in the art. The term “chemotherapy” refers to treatment of a disease characterized by abnormal cell proliferation with chemicals or drugs. The term “immunotherapy” refers to treatment of disease by modulation of the immune system and/or responses. The term “hormonal therapy” refers to treatment of a disease characterized by abnormal cell proliferation with different hormones or their inhibitors. The term “genetic therapy ” refers to treatment of disease characterized by abnormal cell proliferation with compositions containing different genes or gene products, including antisense therapy. The term “subject” refers to the human or animal to whom halofuginone is administered.

According to one aspect the present invention provides a method for increasing the effectiveness of additional known anti tumor treatments, the method comprising the step of co-administering to a subject in need thereof a pharmaceutical composition comprising as an active ingredient a quinazolinone derivative compound having the general formula I: 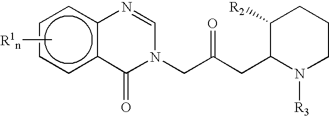
wherein: n=1 -2
R₁ is at each occurrence independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof,
and at least one additional known anti tumor treatment.

According to one currently preferred embodiment, the quinazolinone derivative is halofuginone.

Hereinafter, the term “halofuginone” is defined as a compound having the formula: 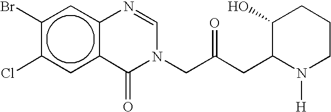
and pharmaceutically acceptable salts thereof.

A composition comprising halofuginone preferably further comprises a pharmaceutically acceptable carrier for the compound.

According to one embodiment, the known anti tumor treatments applied in combination with the quinazolinone compositions of the present invention is selected from the group consisting of radiation therapy, chemotherapy, immunotherapy, hormonal therapy and genetic therapy.

According to one currently preferred embodiment the anti tumor treatment is selected from the group consisting of radiation or chemotherapy.

Some inventors of the present invention have previously disclosed (U.S. Pat. No. 6,420,371 to Pines et al) that halofuginone by itself inhibits tumor progression in vivo. It was suggested that halofuginone mode of action in inhibiting tumor progression may be via inhibiting angiogenesis or via substantially inhibiting deposition of extracellular cell matrix components, or via a combination of both.

The mechanism by which halofuginone enhances the efficacy of chemotherapy or irradiation in treatments of tumor cells is not clear. Without wishing to be bound to a specific mechanism, halofuginone may act by increasing the sensitivity of tumor cells to the toxic effects of ionizing radiation or chemotherapy treatment, although other mechanisms can also be involved.

Some of the most effective and commonly used chemotherapy agents, including but not limited to taxol, gemacetabin, vinca alkaloids and many others, are known to affect cancer cells in a specific stage of the cell cycle. These agents may therefore be described as “cell cycle specific agents”. The cell cycle can be described as a sequence of phases through which the cell proceeds as it proliferates. The phases of this cycle are denoted G1, S, G2 and M, where G1 is the gap preceding synthesis of DNA, S is the phase during which the cell synthesizes DNA, G2 is the gap between the S phase and division or mitosis (M). Cells that are not proliferating may be arrested in a stage referred to as G₀.

It was shown by one of the inventors of the present invention that halofuginone reversibly arrests cells in the G₀/G₁ stage. Upon removal of halofuginone, cells are able to enter the S phase and continue cycling (Nagler et. al. Kidney Int. Vol. 52(1997), pp. 1561-1569). Therefore, the co-administration of halofuginone as a synchronizing agent will sensitize the tumor cells towards a cell cycle specific agent, as defined above. Upon exposure to halofuginone the cell cycle will be arrested, whereas upon its removal the cancer cells will regain their normal cycling. Effectively, this serves to synchronize the cells, thus bringing a larger proportion of the cells to the specific stage of the cell cycle where they will be sensitive to the effects of the chemotherapeutic agent.

According to one embodiment of the present invention the enhancement of the effectiveness of known anti-tumor treatments is obtained by pretreatment with a quinazolinone of general formula I, preferably halofuginone. This is particularly effective when the additional anti-tumor treatment is selected from the group consisting of radiation therapy and chemotherapy.

According to yet another embodiment of the present invention the enhancement of the effectiveness of known anti-tumor treatments is obtained by treatment with a quinazolinone of general formula I, preferably halofuginone, at substantially the same time as the treatment with the additional known anti-tumor treatment. Administration may be in a single composition or in separate compositions as appropriate for the optimal formulation of each agent.

According to another aspect the present invention provides a combined composition for increasing the effectiveness of known anti-tumor treatments comprising a quinazolinone derivative compound having the general formula I: 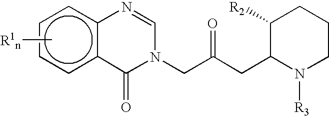
wherein: n=1-2
R₁ is at each occurrence independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof,
further comprising at least one additional anti tumor agent.

According to one currently preferred embodiment of the present invention, the combined composition comprises halofuginone.

Specific non-limiting examples of chemotherapeutic agents that are beneficially administered together with quinazolinone derivatives according to the present invention include, but are not limited to, doxorubicin, daunorubicin, idarubicin, epirubicin, melphalan, dacarbazine, cisplatin, carboplatin and mitomycin.

Additional cancer chemotherapeutic agents suitable for use in combination with the compositions and methods of the present invention may be selected from the following categories: topoisomerase inhibitors, spindle poison vincas: vinblastine, vincristine, vinorelbine (taxol), paclitaxel, docetaxel; alkylating agents: mechlorethamine, chlorambucil, cyclophosphamide, ifosfamide; methotrexate; 6-mercaptopurine; 5-fluorouracil, cytarabine, gemcitabin; podophyllotoxins: etoposide, irinotecan, topotecan, dacarbazin; antibiotics: bleomycin; nitrosoureas: carmustine (BCNU), lomustine; inorganic ions: cisplatin, carboplatin; interferon, asparaginase; hormones: tamoxifen, leuprolide, flutamide, megestrol acetate.

According to yet another aspect the present invention provides the use of a quinazolinone derivative having the general formula I: 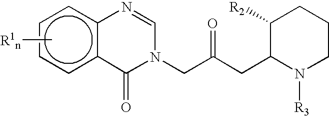
wherein: n=1-2
R₁ at each occurrence is independently a member of the group consisting of hydrogen, halogen, nitro, benzo, lower alkyl, phenyl and lower alkoxy;
R₂ is a member of the group consisting of hydroxy, acetoxy and lower alkoxy; and
R₃ is a member of the group consisting of hydrogen and lower alkenoxy-carbonyl; or pharmaceutically acceptable salts thereof,
in preparation of a medicament for treating a tumor in combination therapy with at least one additional known anti tumor treatment, for improving the effectiveness of the anti tumor treatment.

According to one preferred embodiment, the quinazolinone derivative used in combination therapy is halofuginone.

According to one embodiment, the combined therapy comprises an additional known anti tumor treatment selected from the group consisting of radiation therapy, chemotherapy, immunotherapy, hormonal therapy and genetic therapy.

According to one currently preferred embodiment the combined therapy comprises an additional known anti tumor treatment selected from the group consisting of radiation therapy or chemotherapy.

According to another embodiment, the chemotherapeutic agent is selected from the group consisting of topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, megestrol acetate.

According to another aspect the present invention provides a method for alleviating or preventing the damage induced by radiation therapy, comprising the step of administering to a subject undergoing radiation therapy a therapeutically effective amount of quinazolinone derivative compounds having the general formula I: 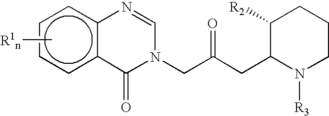
wherein: n=1-2
R₁ which at each occurrence is independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof.

According to one currently preferred embodiment, the quinazolinone compound is halofuginone.

According to one currently preferred embodiment, the quinazolinone composition of the present invention is administered prior to administration of the radiation therapy.

According to one embodiment the compositions of the present invention may be administered orally or parenterally.

Pharmaceutical compositions for oral administration are formulated as aqueous or solid dosage form.

According to one embodiment the pharmaceutical compositions for oral administration are formulated in aqueous form selected from the group consisting of sterile solutions, sterile suspensions, sterile dry soluble lyophilized powders ready for reconstitution by combination with a vehicle just prior to use, sterile emulsions, microemulsions, dispersions, liposomal dosage forms, lipid complexes such as with cholesterol derivatives and phospholipids.

According to one embodiment the solutions and vehicles are selected from the group consisting of aqueous or non-aqueous solutions.

According to one embodiment, the aqueous oral vehicle may further comprise cosolvents such as ethyl alcohol, polyethylene glycol, propylene glycol and mixture thereof.

According to one embodiment the sterile formulation may comprise lyophilized powders ready for reconstitution by aqueous vehicle.

Optionally, at least one additional ingredient selected from the group consisting of, preservatives, antioxidants and tonicity controlling agents can be used.

According to one embodiment the preservatives are selected from the group consisting of benzyl alcohol, methyl paraben, propyl paraben, sodium salts of methyl paraben.

According to one embodiment the tonicity controlling agents are selected from the group comprising of sodium chloride, mannitol, dextrose, glucose, lactose and sucrose.

According to yet another embodiment the pharmaceutical compositions for oral administration are formulated in a solid form selected from the group consisting of tablets, capsules, sachets, powders, granules and lozenges.

According to one embodiment the present invention relates to a solid pharmaceutical formulated as tablets containing in addition to the active compound suitable excipients include, but are not limited to, starches, gum arabic, calcium silicate, microcrystalline cellulose, polyvinylpyrrolidone, cellulose, water, syrup, and methyl cellulose. The formulations can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartarate, succinate, and the like. Other inert fillers which may be used encompass those which are known in the art and are useful in the manufacture of various dosage forms. If desired, the solid pharmaceutical compositions may include other components such as bulking agents and/or granulating agents, and the like. The compositions of the invention can be formulated so as to provide quick, sustained, or delayed release of the active ingredient after administration to the patient by employing procedures well known in the art.

Pharmaceutical compositions for parenteral administration are formulated for intravenous injections, intravenous infusion, intradermal, intralesional, intramuscular, and subcutaneous injections or depots; or they may be administered parenterally by means other than an injection, for example, it could be introduced laparascopically, intravesicularly, or via any orifice not related to the gastrointestinal tract.

According to one embodiment the pharmaceutical compositions for parenteral administration are preferably a formulation selected from the group consisting of sterile solutions ready for injection, sterile suspensions ready for injection, sterile dry soluble lyophilized powders ready for reconstitution by combination with a vehicle just prior to use, sterile emulsions, microemulsions, dispersions, liposomal dosage forms, lipid complexes such as with cholesterol derivatives and phospholipids.

According to one embodiment the solutions and vehicles are selected from the group consisting of aqueous or non-aqueous solutions. In a preferred embodiment the aqueous parenteral solutions and vehicles are selected from the group consisting of sterile water for injection, sodium chloride injection, Ringers injection, isotonic dextrose injection, dextrose and lactated Ringers injection.

According to one embodiment, the aqueous parenteral vehicle may further comprise cosolvents also referred to as water miscible solvents such as ethyl alcohol, polyethylene glycol, propylene glycol and mixture thereof.

According to one embodiment the sterile injection may comprise lyophilized powders ready for reconstitution by aqueous vehicle. Such lyophilized powders comprising quinazolinone derivative and a solid pharmaceutically acceptable buffering agent or a water-soluble organic acid. The buffering agents or organic acids used in the composition may be any non-toxic buffering agent or organic acid approved for parenteral use.

Optionally, at least one additional ingredient selected from the group consisting of, preservatives, antioxidants and tonicity controlling agents can be used.

According to one embodiment the preservatives are selected from the group consisting of benzyl alcohol, methyl paraben, propyl paraben, sodium salts of methyl paraben.

According to one embodiment the tonicity controlling agents are selected from the group comprising of sodium chloride, mannitol, dextrose, glucose, lactose and sucrose.

Although the specific quinazolinone derivative “halofuginone” is referred to throughout the specification, it is understood that other quinazolinone derivatives may be used in its place, these derivatives having the general formula I: 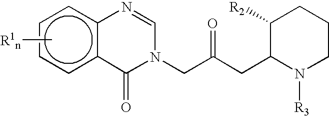
wherein: n=1-2
R₁ is at each occurrence independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or pharmaceutically acceptable salts thereof.

As previously disclosed by inventors of the present invention (U.S. Pat. No. 6,420,371 to Pines et. al; U.S. patent application Ser. No. 09/762715 to Pines et. al), halofuginone activity in vivo cannot always be predicted from its activity in vitro. Therefore, there is need to examine the capability of halifuginone to enhance the efficacy of known anti tumor treatments in an in vivo model, as described in greater details in the Examples below.

While the invention will now be described in connection with certain preferred embodiments in the following figures and examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following figures and examples which include preferred embodiments will serve to illustrate the practice of this invention, it being understood that the particulars shown are by way of example and for purposes of illustrative discussion of preferred embodiments of the present invention only, and are presented in the cause of providing what is believed to be the most useful and readily understood description of formulation procedures as well as of the principles and conceptual aspects of the invention.

### EXAMPLES

### Example 1

### The Effect of Combined Treatment of Halofuginone and 1,3-bis(2-chloroethyl)-1-nitrosourea (BCNU)

Halofuginone was tested in the human T98G glioblastoma xenograft implanted subcutaneously or intracranially. Mice were implanted with the human T98G glioblastoma tumor cells subcutaneously in a thigh or intracranially.

Halofuginone was administered orally by gavage at dose levels of 0.1,0.2, and 0.5 mg/kg/day, once per day, on days 4 through 34 days post tumor implantation. Each group contained 5 mice.

The endpoint for the subcutaneous tumor was tumor growth delay while the endpoint for the intracranial tumor was increase-in-lifespan (survival).

There was no effect of halofuginone on the body weight of the animals.

TABLE 1
Response of the Human T98G Glioblastoma Multiforme to treatment
with BCNU along with halofiginone

| | TUMOR GROWTH | |
|---|---|---|
| | DELAY | SURVIVAL |
| TREATMENT GROUP | Days (sc tumor) | Days (ic tumor) |
| CONTROL | 0 | 64 ± 10 |
| No treatment | | |
| BCNU (15 mg/kg), ip, | 3.9 ± 0.4 | 97 ± 21 |
| days 7, 9, 11 | | |
| BCNU + 0.1 mg/kg | 6.4 ± 0.8 | 103 ± 29 |
| halofiginone | | |
| BCNU + 0.2 mg/kg | 11.1 ± 1.0 | 148 ± 4 |
| halofiginone | | |
| BCNU + 0.5 mg/kg | 12.2 ± 1.6 | 119 ± 21 |
| halofiginone | | |

Combination of halofuginone with BCNU elevated dramatically the tumor growth delay from 3.8 days to 12 days.

In the intracranial model halofuginone in combination with BCNU significantly increased the life span of mice compared to BCNU alone. The pharmacological optimal dose of 0.2 mg/kg body weight prolonged the survival beyond the scope of the study (animals sacrificed in good health after 150 days).

Both effects were dose dependent.

### Example 2

### Halofuginone Induces Cell Cycle Arrest in Rabbit Aortic Smooth Muscle Cells (SMC)

Experiments were conducted to determine the specific phase of the cell cycle in which SMC treated with halofuginone were arrested.

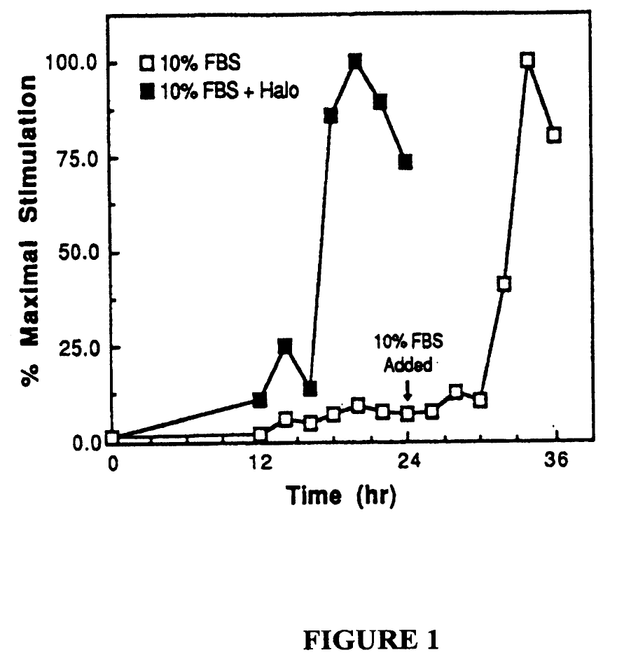

As determined by [³H]-thymidine incorporation, addition of 10% FBS to growth-arrested, quiescent SMC promotes entry of the cells to S phase after a G₁ period of 16 hours. Maximal DNA synthesis was seen 20 hours after serum-stimulation (FIG. 1).

When halofuginone (10⁻⁷ M) was added with 10% FBS, only low levels of [³H]-thymidine incorporation were observed (FIG. 1). It was next determined whether halofuginone arrested proliferation at a specific stage in the cell cycle. For these experiments, quiescent SMCs were kept in 10% FBS plus 10⁻⁷ M halofuginone for 24 hours. The cultures were then washed and placed in 10% FBS with [³H]-thymidine and without halofuginone. At various times after halofuginone removal, the cells were harvested and thymidine incorporation was determined. When halofuginone-treated cells were released from growth-arrest, there was a lag of 4-6 hours before initiation of DNA synthesis, which peaked by 10 hours (FIG. 1).

Since quiescent G₀-arrested SMCs require a minimum of about 16 hours to pass through G₀, pretreatment with 10% FBS plus halofuginone permitted cell cycle progression to a point about 4-6 hours from S phase. Thus, in the continual presence of halofuginone, SMC progress into G₁ and reversibly arrest at late G₁ phase.

### Example 3

### Halofuginone Arrest Rat Mesangial Cells (RMC) in G₀/G₁ Phase

Further experiments were conducted to determine whether halofuginone arrests mesangial cell proliferation at a specific phase of the cell cycle.

For this purpose sub confluent RMC's were kept in 10% FCS in the absence or presence of 150 ng/ml halofuginone for 24 hours. The cells were then harvested, stained with propidium iodide and analyzed by FACScan. The percentage of cells progressing into G₂/M phase was reduced by halofuginone from 20% to 7%. The percentage of cells in G₀/G₁ was increased from 38% in the absence of halofuginone to 65% in the presence of halofuginone. These results indicate that in the presence of halofuginone, a large proportion of the mesangial cells are arrested in the G₀/G₁ phase.

### Example 4

### Combination Treatment of Halofuginone and Melphalan on Multiple Myeloma Cells

Materials and Methods

Multiple Myeloma (MM) cell line U266B1 was purchased from ATCC (TIB-196).

WST-1 reagent (Roche 1 644 807)

Melphalan-SIGMA M2011

1. WST—Viability Test

Cells were grown in RPMI supplemented with 20% FCS. Cells were seeded into 96 well plates (30K cell/well) with various concentrations of halofuginone or melphalan. After incubation period WST reagent was added to wells and cells were incubated for about 24 hours at 37° C., 5% CO₂. Absorbance was measured at 440 nm using scanning multi-well spectrophotometer (ELISA reader).

2. Cell Cycle Analysis

Cells (10⁶) were incubated for 48 hours with different concentrations of halofuginone. The cells were permeabilized with 70% ethanol in PBS for 30 minutes at 4° C., and then incubated with 0.5 ml of a 50 μg/ml Propidium Iodide solution containing 20 U/ml RnaseA for 30 minutes. Cells were analyzed by flow cytometry.

Results

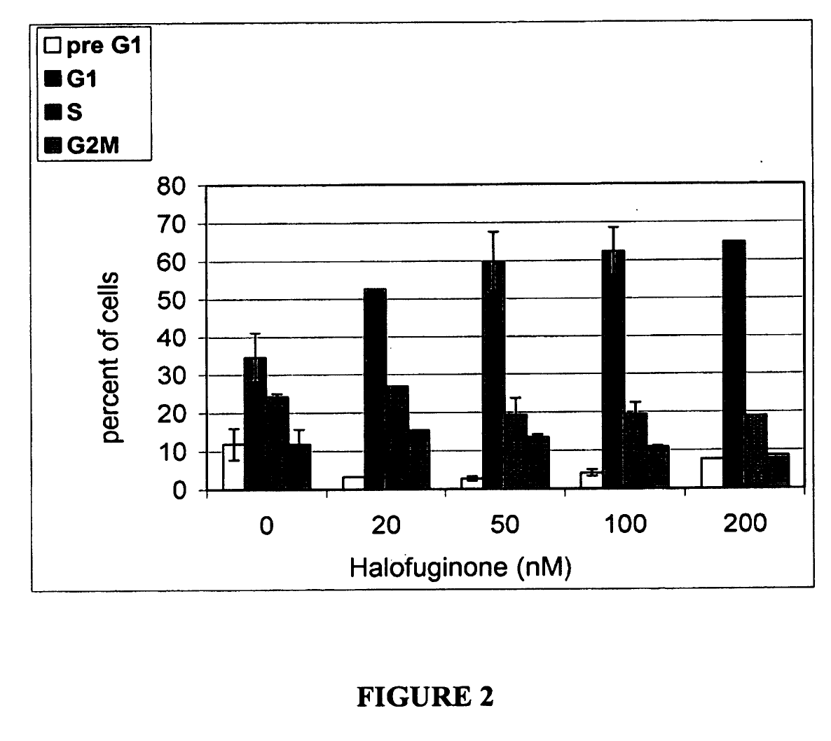

As shown in FIG. 2, treatment of U266 cells with halofuginone caused elevation in the number of cells arrested in G1 phase.

As a second step, the effect of pretreatment with halofuginone on the sensitivity of MM cells to melphalan, a known anti-tumor treatment, was examined.

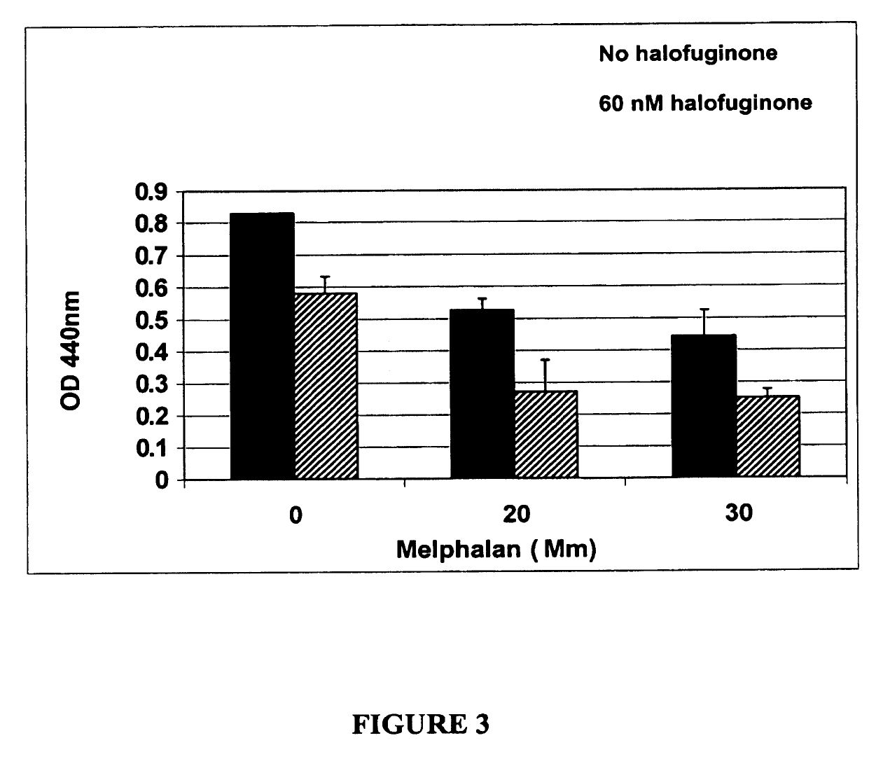

Cells (30K) were treated for 48 hours with 60 nM halofuginone with subsequent treatment with melphalan for 72 hours in various concentrations. At the end of the incubation, cell viability was measured by WST-viability test. Sequential treatment of halofuginone and melphalan was more effective than treatment with melphalan alone (FIG. 3), thus demonstrating the synergistic effect between halofuginone and melphalan treatments. It is suggested that the synchronization of the cells in pre-G1 phase of the cell cycle rendered them more sensitive to the melphalan treatment.

### Example 5

### Halofuginone Decreased Fibrosis Induced by Radiation

Mice were injected intraperitoneally once daily with 1-5 μg/mouse of halofuginone, for a period of 4 months.

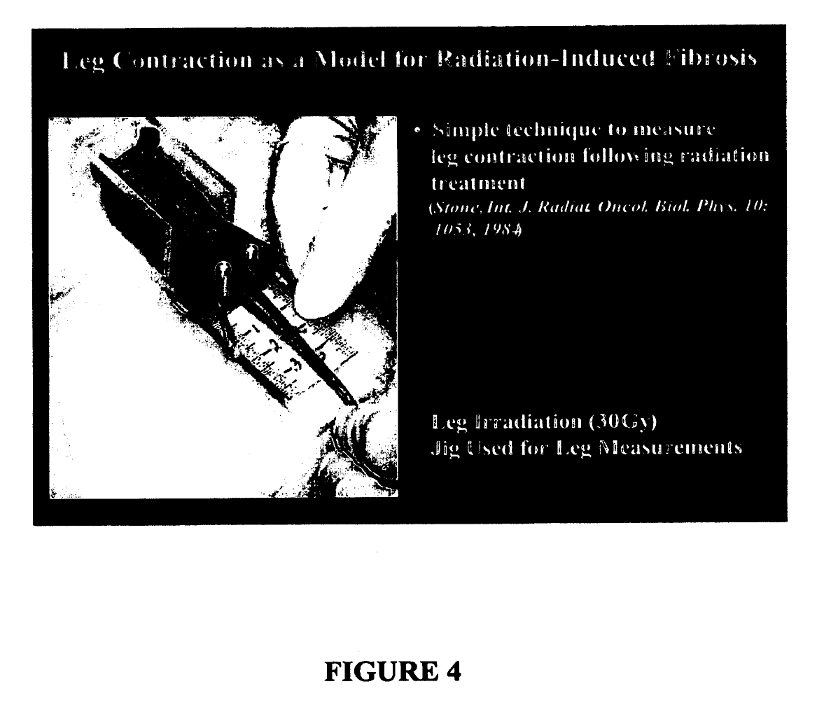

The right leg only of each animal was radiated with 35Gy or 45Gy. In the control group mice did not receive halofuginone and the right leg was radiated with 35Gy or 45Gy. Leg contraction was measured as demonstrated in FIG. 4. Measurements were taken within time periods of 2 to 4 months after radiation.

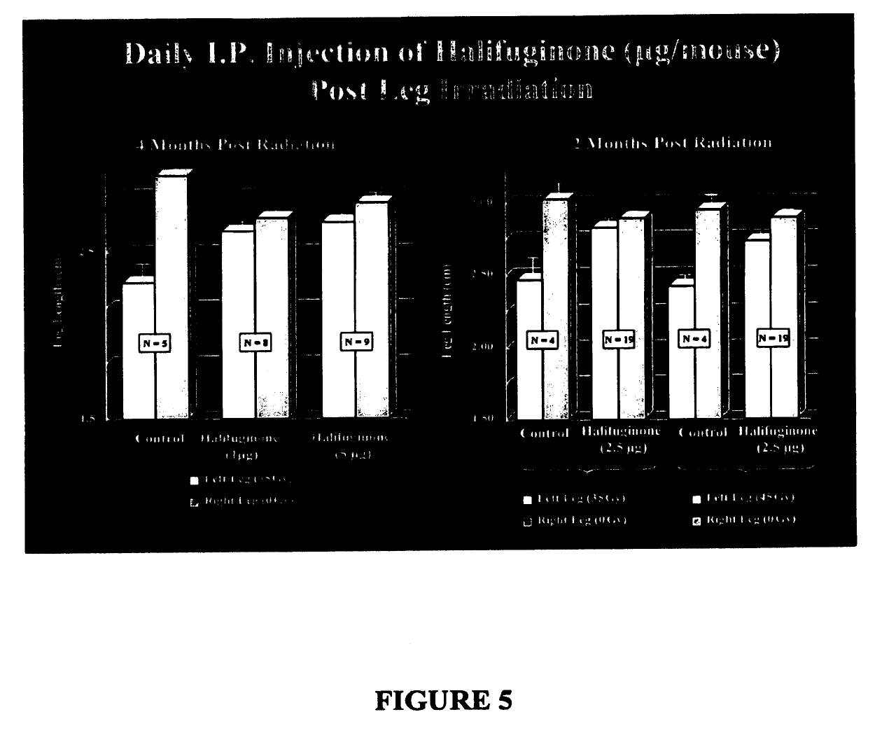

As shown by FIG. 5, dramatic decrease in the “leg length difference” between the right and left leg is observed in halofuginone treated animals. The effect of halofuginone can be observed post radiation at 2 and 4 months, at 35Gy and 45 Gy radiation and at all of the used halofuginone concentrations (1-5μg/mouse).

In general, it can be concluded that halofuginone reduced the radiation effects in this in-vivo model, as the irradiated leg of mice that received halofuginone was definitely less stiff, and the skin was less dry in comparison to mice that did not receive halofuginone.

### Example 6

### Halofuginone Acts as a Radiation Sensitizer

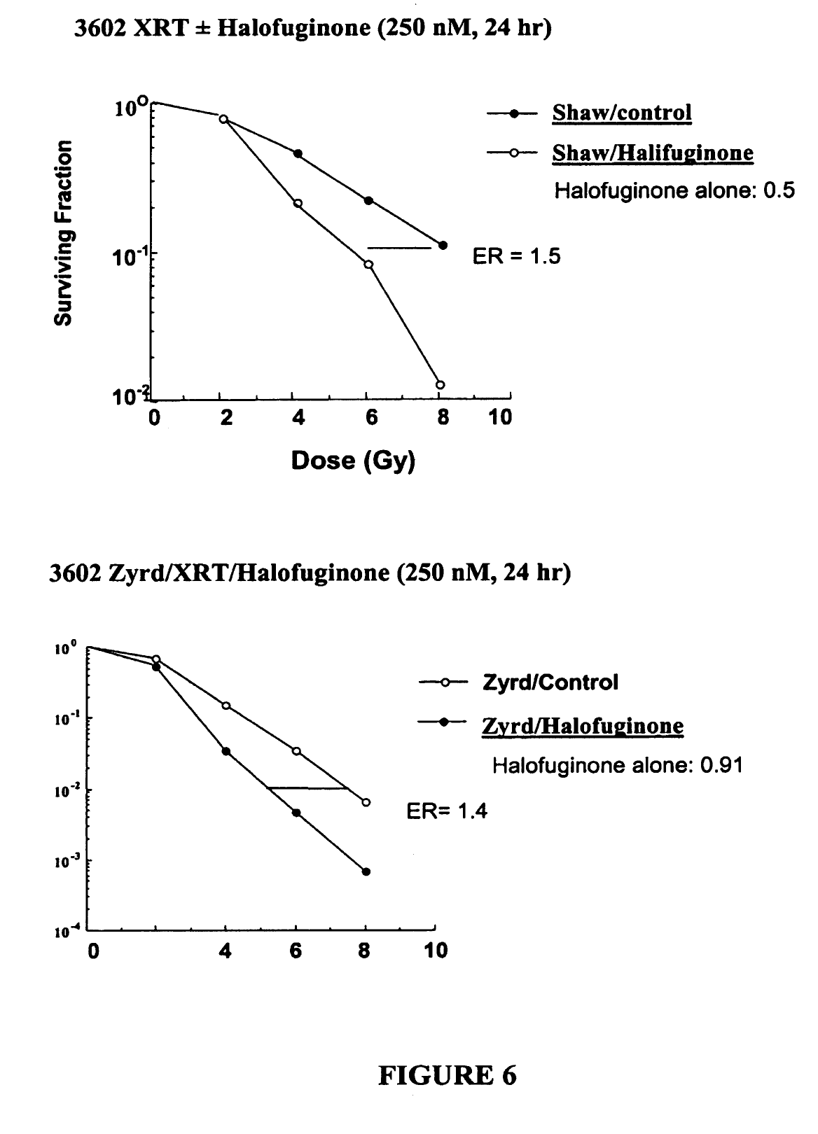

Two pancreatic cancer cell lines: 3602 Xrt and 3602 Zyrd/Xrt were incubated with or without 250 nM halofuginone for 24 hr, than radiated with 0-8 Gy. Survival fraction of the cell was determined. As shown in FIG. 6, halofuginone caused a decrease in the survival fraction of approximately 50%. These results confirm the observation that halofufinone increase the sensitivity of the tumor cells to the anti-tumorigenic treatment of radiation.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever chemical structure, or whatever function, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

1. A method for improving the effectiveness of an anti-tumor treatment comprising the step of co-administering to a subject in need thereof a pharmaceutical composition comprising as an active ingredient a quinazolinone derivative compound having the general formula I: 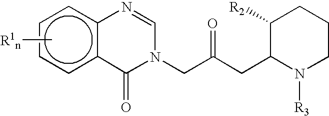
wherein: n=1-2
R₁ at each occurrence is independently selected from the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or
pharmaceutically acceptable salts thereof,
and at least one additional anti tumor treatment.

2. The method according to claim 1 wherein the subject is human.

3. The method according to claim 1 wherein the administration of the quinazolinone composition is prior to the administration of the at least one additional anti-tumor treatment.

4. The method according to claim 1 wherein the administration of the quinazolinone composition is substantially at the same time as the administration of the at least one additional anti-tumor treatment.

5. The method according to claim 4 wherein the co-administration is in a single pharmaceutical composition.

6. The method according to claim 4 wherein the co-administration is in separate pharmaceutical compositions.

7. The method according to any one of claims 1-4 wherein the anti tumor treatment is radiation therapy.

8. The method according to any one of claims 1-6 wherein the anti tumor treatment is chemotherapy.

9. The method according to any one of claims 1-6 wherein the anti tumor treatment is selected from the group consisting of immunotherapy, hormonal therapy and genetic therapy.

10. The method according to claim 1 wherein the improvement in effectiveness is achieved by enhancement of cellular sensitivity to the anti tumor treatment.

11. The method according to any one of claims 1-10 wherein the compound of formula I is halofuginone or a pharmaceutically acceptable salt, solvent or hydrate thereof.

12. The method according to claim 8, wherein the additional agent used for chemotherapy is selected from the group consisting of topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, megestrol acetate.

13. A combined pharmaceutical composition comprising as an active ingredient a quinazolinone derivative compound having the general formula I: 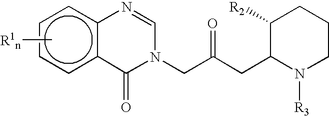
wherein: n=1-2
R₁ at each occurrence is independently selected from the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or
pharmaceutically acceptable salts thereof, and at least one pharmaceutically acceptable carrier or diluents;
further comprising at least one additional anti tumor agent.

14. The pharmaceutical composition according to claims 13 wherein the compound of formula I is halofuginone or a pharmaceutically acceptable salt, solvent or hydrate thereof.

15. The pharmaceutical composition according to claim 13 wherein the anti tumor agent is a chemotherapeutic agent.

16. The pharmaceutical composition according to claims 15, wherein the chemotherapeutic agent is selected from the group consisting of topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, megestrol acetate.

17. Use of a quinazolinone derivative compound having the general formula 1: 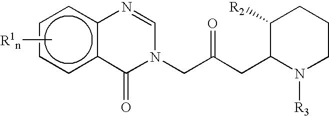
wherein: n=1-2
R₁ at each occurrence is independently selected from the group consisting of the hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or
pharmaceutically acceptable salts thereof,
in the preparation of a medicament for treating a tumor in combination therapy with at least one additional anti tumor treatment, thereby improving the effectiveness of the anti tumor treatment.

18. Use according to claim 17, wherein the additional anti tumor treatment is radiation therapy.

19. Use according to claim 17, wherein the additional anti tumor treatment is chemotherapy.

20. Use according to claim 17, wherein the additional anti tumor treatment is selected from the group consisting of immunotherapy, hormonal therapy and genetic therapy.

21. Use according to claim 17, wherein the improvement is achieved by enhancement of cellular sensitivity to the anti tumor treatment.

22. Use according to claim 17 wherein the compound of formula I is halofuginone or a pharmaceutically acceptable salt, solvent or hydrate thereof.

23. Use according to claim 19 wherein the chemotherapeutic agent is selected from the group consisting of topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, megestrol acetate.

24. The pharmaceutical composition of any one of claims 13-16 formulated in a form suitable for administration of the composition orally or parenterally.

25. The pharmaceutical composition according to claims 24 wherein the formulation for parenteral administration is selected from a dosage form suitable for intravenous injections, intravenous infusion; intradermal, intralesional, intramuscular, and subcutaneous injections or depots; for administration parenterally by means other than injection, laparascopically, intravesicularly, or intralesionally.

26. The pharmaceutical composition according to claim 24 formulated for oral administration in a form selected from a powder, granules, suspensions or solutions in water or non aqueous media, sachets, capsules or tablets.

27. A method for alleviating or preventing the damage induced by radiation therapy comprising the step of administering to a subject undergoing radiation therapy a pharmaceutical composition comprising as an active ingredient a quinazolinone derivative compound having the formula I: 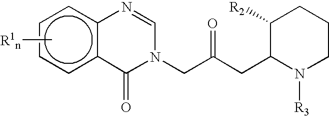
wherein: n=1-2
R₁ at each occurrence is independently a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; or
pharmaceutically acceptable salts thereof,
further comprising a pharmaceutically acceptable carrier.

28. The method according to claim 27 wherein the compound according to formula I is halofuginone a pharmaceutically acceptable salt, solvent or hydrate thereof.

29. The method according to claim 27 wherein the administration is prior to the administration of radiation therapy.
